(19) United States
(12) Patent Application Publication
(10) Pub. No.: US 2002/0076822 A1
(43) Pub. Date: Jun. 20, 2002

(54) Fluorescence-based method for detecting of basic gases

(76) Inventors: Igor A. Levitsky, Fall River, MA (US); Sergei G. Krivoshlykov, Shrewsbury, MA (US)

Correspondence Address: Sergei G. Krivoshlykov, ALTAIR Center, LLc, 1 Chartwell Circle, Shrewsbury, MA, 01545, US

(21) Appl. No.: 09/736,881

(22) Filed: Dec. 14, 2000

(51) Int. Cl. G01N 33/00; G01N 31/22

(52) U.S. Cl. 436/104; 436/100; 436/103; 436/166; 436/167; 436/169; 436/172

(57) ABSTRACT

A fluorescence-based method for highly sensitive and selective detection of molecules of basic gases, such as dimethyl methylphosphonate (DMMP), Sarin, Soman and other chemical warfare agents, is proposed. The method employs the effect of strong fluorescence change in a solvatochromic dye isolated in a matrix of the hydrogen bond acidic polymer. In one preferred embodiment the dye and polymer matrix are chosen such that the hydrogen-bond interaction between them results in depression of the fluorescence yield of the sensitive material prior its interaction with the molecules of basic gases. The interaction between the molecule of basic gases and the acidic polymer matrix breaks the hydrogen bond of the dye with the polymer matrix “releasing” the dye and returning it back into the state with a low solute-solvent interaction. That results in strong enhancement of the dye florescence quantum yield and shift of the dye fluorescent spectrum in the direction of shorter wavelengths. The method can be used in fluorescence chemical sensors of basic gases for different applications including environmental monitoring, control of industrial processes and medicine.

### BACKGROUND OF THE INVENTION

1. Field of the Invention

The present invention relates to the methods for detection of molecules of basic gases, and particularly to the fluorescence-based method of detection employing solid films of sensory chromophores isolated in a hydrogen-bond acidic polymer matrix. In one preferred embodiment, a solvatochromic dye isolated in the acidic polymer matrix is chosen such that the hydrogen-bond interaction between the dye and polymer results in depression of the fluorescence yield of the sensitive material prior its interaction with the molecules of basic gases and the interaction between the molecule of basic gases and the acidic polymer matrix breaks the hydrogen bond of the dye with the polymer matrix “releasing” the dye and returning it back into the state with a low solute-solvent interaction resulting in strong enhancement of the dye florescence quantum yield and shift of the dye fluorescent spectrum in the direction of shorter wavelengths. In another preferred embodiment, the sensitive material is incorporated into an optical fiber system enabling efficient excitation of the dye and collecting the fluorescent signal form the sensitive material on the remote end of the system.

The method can be used in all applications where highly sensitive detection of basic gases, such as dimethyl methylphosphonate (DMMP), Sarin, Soman and other chemical warfare agents having basic properties, is required, including environmental monitoring, chemical industry and medicine.

2. Information Disclosure Statement

Today, there is a high demand for chemical sensor for detecting low concentration levels of analytes present in the liquid and gaseous phase. Selectivity to target molecules is also highly desired. Traditional methods of quantitative detection of analytes based on gas chromatography and mass spectrometry require complex laboratory equipment. Among modern approaches for the real time monitoring of gaseous analytes, mainly three kinds of sensing elements have been investigated: microelectrodes, quartz crystal microbalance and surface acoustic wave devices. Generally all these methods are based on detection of only one parameter—signal intensity. Therefore reliable analyte identification requires significant increasing the number of individual sensors in the detector array.

Meanwhile, optical chemosensors, especially fluorescence-based chemosensors can provide many kinds of complex information, including changes in intensity, wavelengths and spectral shape, fluorescence lifetime. Hence such promising approach allowing detection of many parameters simultaneously should make possible fabrication of highly sensitive, robust, multi-analyte-detecting arrays with fewer independent sensors. Moreover, the possibility of remote sensing using optical fluorescence technique offers many serious advantages over other traditional methods of real-time monitoring of toxic gases and pollutants.

Dr. Grate and coworkers at PNNL are developing a versatile approach for the synthesis of special polymers for sorption of basic vapors. U.S. Pat. No. 6,015,869 describes the polymers having strong hydrogen bond acidic properties. So far these polymers have been applied only to detection of basic vapors (simulants of chemical warfare agents) employing surface acoustic waves or potentiometric methods. Nothing has been reported yet about the design of fluorescence chemosensors based on emissive solvatochromic dye isolated in the hydrogen bond acidic polymer matrix.

The present invention suggests new approach to the highly sensitive and selective detection of basic gases using fluorescence properties of the solvatochromic dye isolated in the hydrogen bond acidic polymer matrix. It is based on recently demonstrated new effect of strong changing the fluorescence emission in the presence of molecules of basic gases. In one preferred embodiment the fluorescent sensitive material is incorporated into a fiber-optical system allowing remote monitoring of large contaminated area.

The method can be used in all applications where highly sensitive detection of basic gases, such as dimethyl methylphosphonate (DMMP), Sarin, Soman and other chemical warfare agents having basic properties, is required, including environmental monitoring, chemical industry and medicine.

### OBJECTS AND SUMMARY OF THE INVENTION

It is an object of the present invention to improve performance of chemical sensors of basic gases by employing the effect of fluorescence change of the solvatochromic dye isolated in the hydrogen bond acidic polymer matrix in the presence of target molecules.

Another object of the invention is to provide a simple and efficient method of detection of the fluorescence signal by measuring enhancement of the fluorescence in the presence of target molecules.

A further object is to provide a selection of efficient sensitive fluorescent material comprising a film of Nile Red (NR) dye having functional basic group and isolated in BSP3 polymer matrix that is a strong hydrogen bond acidic polymer.

Another object is to provide efficient method of processing the fluorescent signal from the sensitive material.

Still another object is to provide a possibility of remote monitoring of large contaminated area by incorporating the sensitive fluorescent material into an optical fiber system.

An additional object of the invention is to provide a method for fabrication of the fiber-optic fluorescence sensors achieving efficient excitation of the sensitive material and efficient collecting the fluorescence signal.

Briefly stated, the present invention provides a method of detection of different gases having basic properties, such as dimethyl methylphosphonate (DMMP), Sarin, Soman and other chemical warfare agents. The method employs the effect of strong fluorescence change in a solvatochromic dye isolated in a matrix of the hydrogen bond acidic polymer. In one preferred embodiment the dye and polymer matrix are chosen such that the hydrogen-bond interaction between them results in depression of the fluorescence yield of the sensitive material prior its interaction with the molecules of basic gases. The interaction between the molecule of basic gases and the acidic polymer matrix breaks the hydrogen bond of the dye with the polymer matrix “releasing” the dye and returning it back into the state with a low solute-solvent interaction. That results in strong enhancement of the dye florescence quantum yield and shift of the dye fluorescent spectrum in the direction of shorter wavelengths. The method can be used in fluorescence chemical sensors of basic gases for different applications including environmental monitoring, control of industrial processes and medicine.

The above, and other objects, features and advantages of the present invitation will become apparent from the following description read in conjunction with the accompanying drawings.

### BRIEF DESCRIPTION OF DRAWINGS

FIG. 1 Shows the structure of Nile Red dye.

FIG. 2 Shows repeat units of PSFA, BSP3 and PMMA polymers.

FIG. 3 Illustrates Nile Red fluorescence spectra before (1) and after (2) DMMP exposure in BSP3 (a), PSFA (b) and PMMA (c) spin cast films. Curves (3) are curves (1) ×5 and ×10 for PSFA and BSP3, respectively.

FIG. 4 Illustrates time traces of NR fluorescence in BSP3, PSFA and PMMA films at λₑₓ=550 nm and λ_{det}=635 nm. Solid and dotted lines correspond to ‘thick’ films (1000 rpm) and ‘thin’ films (3000 rpm), respectively (a). Time traces of NR fluorescence in BSP3 film at different excitation and monitoring wavelengths (b).

FIG. 5 Shows visible absorption spectra of NR in BSP3 film before and after saturated DMMP exposure.

FIG. 6 Illustrates time trace of the NR fluorescence signal in a BSP3 polymer matrix (λₑₓ=530 nm, λ_{det}=590 nm) under alternating exposures to diluted DMMP vapors and clean nitrogen gas.

FIG. 7 Illustrates sensor selectivity without (a) and with (b) normalization on the concentration of saturated vapors. Signal was detected after 300 sec under vapor exposure. The sample is NR in BSP3 film (λₑₓ=530 nm, λ_{det}=590 nm).

FIG. 8 Shows double tapered fiber probe.

### DESCRIPTION OF PREFERRED EMBODIMENTS

The invention provides a new method for detection of basic gases employing fluorescence of a solvatochromic dye isolated in a matrix of the hydrogen bond acidic polymer. The polymer matrix and solvatochromic dye are chosen such to maximize the influence of the molecules of the basic gases under detection on the fluorescence light signal from the sensitive material. The proposed general of detection of molecules of basic gases can be used for the design of many different chemical sensors and fluorescence-based devices.

In one preferred embodiment the dye and polymer matrix are chosen such that the hydrogen-bond interaction between them results in depression of the fluorescence yield of the sensitive material prior its interaction with the molecules of basic gases. Then interaction between the molecule of basic gases and the acidic polymer matrix breaks the hydrogen bond of the dye with the polymer matrix “releasing” the dye and returning it back into the state with a low solutesolvent interaction. That results in strong enhancement of the dye florescence quantum yield and shift of the dye fluorescent spectrum in the direction of shorter wavelengths.

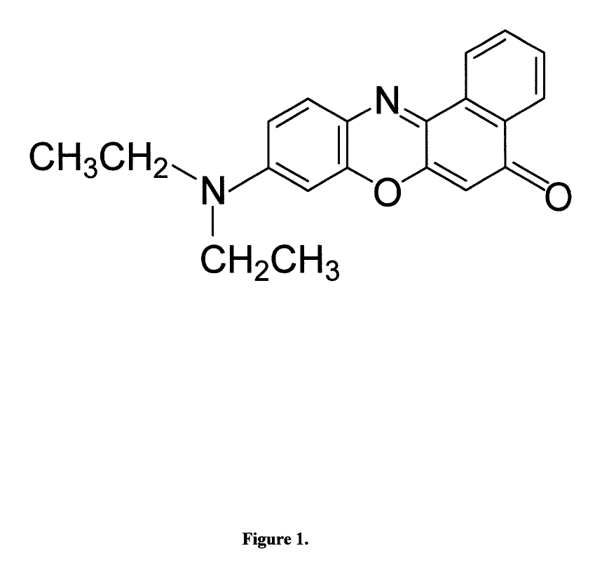

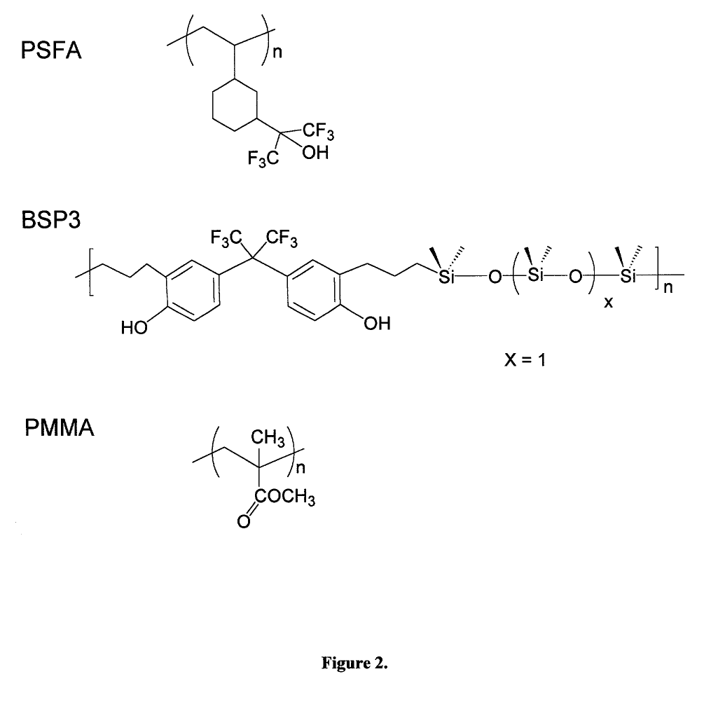

In the preferred embodiment shown in FIG. 1 a solvatochromic dye Nile Red (NR) was employed as an active emissive element. Three different polymers BSP3, PSFA and PMMA shown in FIG. 2 were tested as a matrix for NR. Two of them (BSP3 and PSFA) had strong hydrogen-bond acidic properties. PMMA that does not have hydrogen-bond groups was used as a reference.

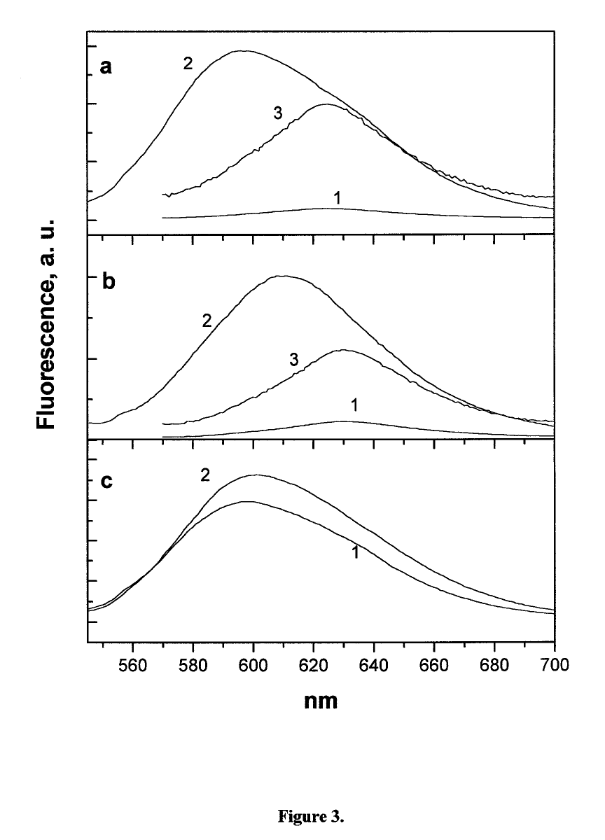

FIG. 3 demonstrates the pronounced NR fluorescence enhancement and the spectral blue shift under exposure to dimethyl methylphosphonate (DMMP) saturated vapors for BSP3 and PSFA matrixes and rather small changes without spectral shift for PMMA matrix. DMMP is a strong basic vapor usually employed as a simulant of chemical warfare agents (CWA), such as Sarin, Soman and other basic CWA agents. The observed strong changes in the dye emission is associated with a competition between NR and DMMP interaction with BSP3 and PSFA polymers. NR forms hydrogen bonds with these polymers resulting in the low quantum yield and the red shift. Since solute DMMP is a strong basic vapor it effectively interacts with hydrogen bond acidic BSP3 and PSFA polymers. Thus, DMMP molecules having higher binding affinity to BSP3 and PSFA than NR breaks NR hydrogen bonds “releasing” dye and turning it back into the states with a low solute-solvent interaction. Such states have more intensive emission and their spectrum is blue shifted with respect to that of initial states. The case of low interaction between the dye and polymer is realized for PMMA matrix. This is consistent with a small change in NR fluorescence under DMMP exposure.

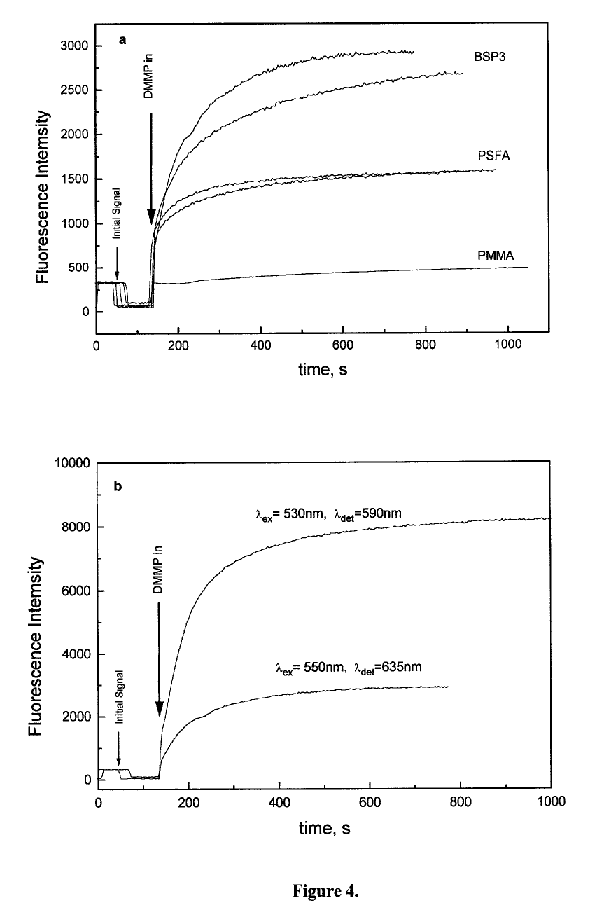

Nile Red fluorescence time trace shows dependence on micro-environment, film thickness and wavelengths of monitoring and excitation (FIG. 4). BSP3 matrix provides higher response signal than PSFA matrix (FIG. 4a). The fluorescence change for PMMA polymer is almost negligible with respect to BSP3 and PSFA polymers.

The better response of BSP3 is associated with both the higher hydrogen bond acidity and the lower T_{g} value in comparison to PSFA polymer. The latter provides relatively big dynamic cavities in the polymer film and facilitates the analyte diffusion. We found confirmation of that in the character of time traces for different film thickness.

There is no difference between relatively thick (540 Å) and thin (380 Å) films in the case of PSFA. However, the response signal for thick (550 Å) BSP3 film is higher then for thin film (330 Å). It means that efficient DMMP permeability in BSP3 polymer film (T_{g} is lower than room temperature) induces the fluorescence enhancement through all film thickness providing higher response for thick films. In contrary, the relatively low DMMP permeability in PSFA polymer films (T_{g} is higher than room temperature) gives DMMP absorption in the layer near film surface. Therefore the fluorescence signal does not depend on the film thickness. Thus, performance of the optical sensor fabricated from BSP3 polymer can be considerably improved by optimizing the thickness.

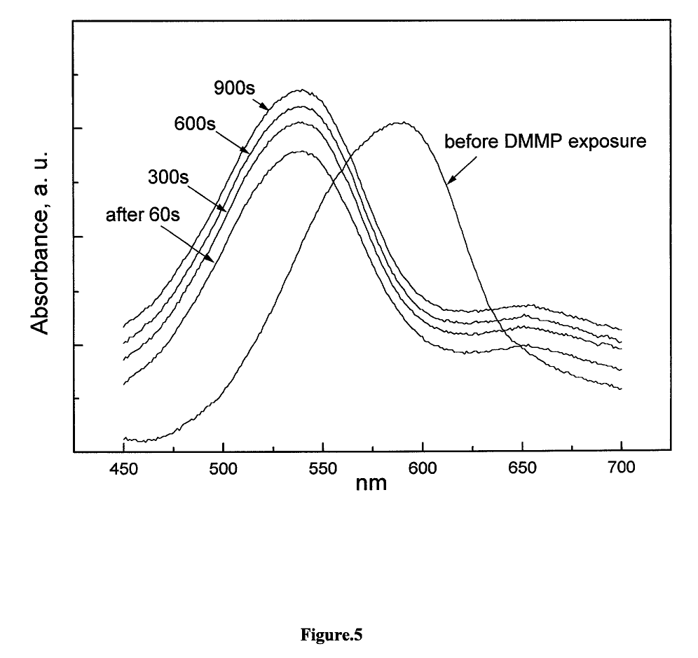

Optimal sensor sensitivity can be attained also by choosing the appropriate excitation (λₑₓ) and monitoring (λ_{det}) wavelengths (FIG. 4b). In one preferred embodiment, the optimal λₑₓ and λ_{det} values are shifted to the shorter wavelengths with respect to the position of maximum of UV-Vis and fluorescence bands before DMMP exposure since NR interaction with DMMP leads to the blue shift of these spectra. The series of NR UV-Vis spectra under DMMP exposure are shown in FIG. 5. Thus, the spectral shift provides efficient amplification of the response signal.

In an additional preferred embodiment, detecting and processing of the fluorescent light signal from the sensitive material is performed for both the spectral shift of dye fluorescence and absorption spectrum. This maximizes the sensor sensitivity and selectivity to the molecules of basic gases under detection.

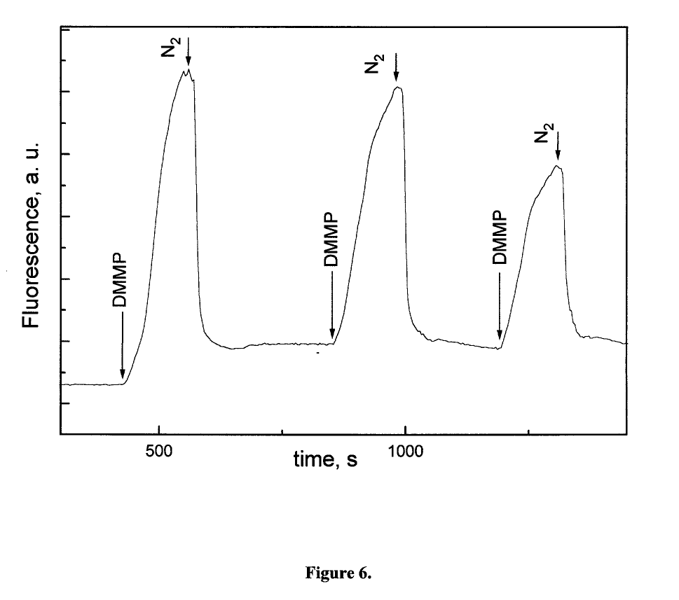

Sensitivity of NR fluorescence response was tested out under exposure to DMMP diluted vapors. Typical time trace of NR emission in BSP3 film at switching DMMP vapor (diluted in 60 times against saturated vapor) to nitrogen is shown in FIG. 6. Dilution of the DMMP saturated vapor in 60 times corresponds to the concentration of about 20 ppm. This value was calculated from the concentration of saturated DMMP - 5960 mg/m³, FW_{DMMP}=124 and the number of Mol in 1 m³ at normal conditions. The peak intensity in FIG. 6 is attained in 100s and it can be higher for longer time of exposure. Also, we examined the low detection limit and found 20-30% change in the fluorescence intensity at DMMP concentration of about 100 - 200 ppb. It follows from FIG. 6 that the sensing material demonstrates quick response on the analyte vapor and almost full and fast recovery after clean gas exposure.

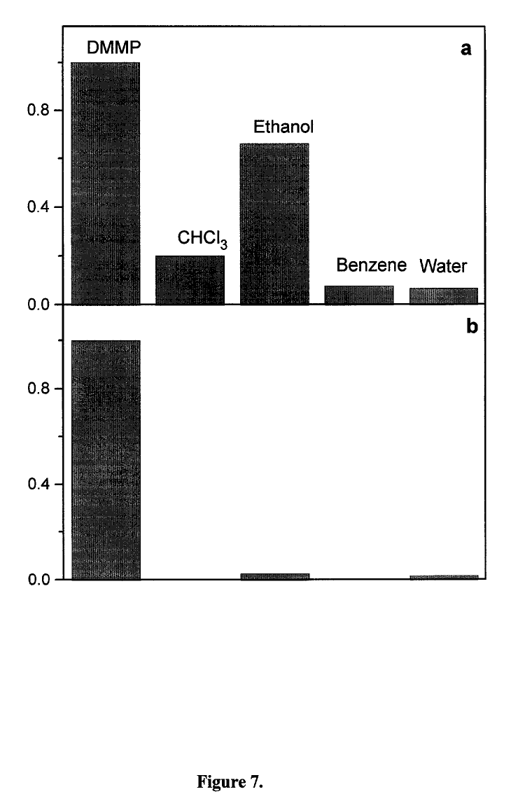

Selectivity of NR/BSP3 films was tested using the common organic vapors and water as interferants to DMMP. These results are presented in FIG. 7. Correct comparison with the sensitivity to DMMP can be made only in the case when all vapors are at equal concentration. After normalization of the response to the saturated concentration (FIG. 7b) we found that sensitivity to DMMP exceeds that to water, benzene, ethanol and chloroform in 58, 750, 37 and 1000 times, respectively.

In another preferred embodiment the proposed method of detection of molecules of basic gases employes sensitive NR/BSP3 compound incorporated into a waveguide or into a fiber-optic system having a flexible fiber-optic probe exhibiting enhanced sensitivity and improved reliability, yet low in cost. In one configuration, the fluorescent sensitive material is excited with a compact blue laser diode or LED generating, for example, at wavelength 405-430 nm.

In still another embodiment the light delivery and collection is performed using a U-bent fiber. It is known that due to sharp bending of the multimode fiber with radius of bending of about 1-2 mm the light escapes from the fiber core into its low-index cladding. After Fresnel reflection at the interface between the cladding and an external low-index media (or air), almost 80% of the light returns back into the fiber core. If the bent region of the fiber is coated with a sensitive fluorescent film, then the returned light will contain also some part of the fluorescent signal. In spite of very simple and cost-efficient design of such a fiber probe it does not provide maximum sensitivity. Length of the bent fiber region is relatively small resulting in a weak fluorescence response signal. Such fiber probe can be used in the applications that do not require very high sensitivity. In order to enhance the sensitivity one should increase the distance of light propagation in the fluorescence film. This is achieved, for example, by removing fiber cladding in the U-bent region (polishing it down to the fiber core) and depositing the fluorescent sensitive material directly on the fiber core.

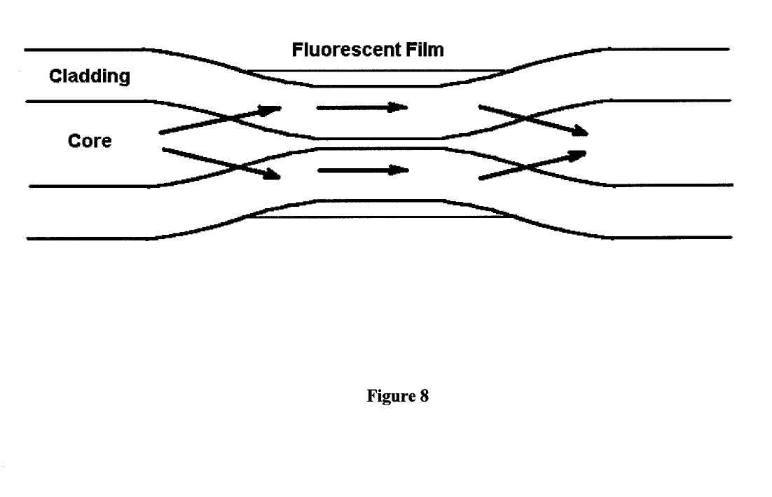

In another preferred embodiment shown in FIG. 8 the light propagation distance of the fluorescent material is increased by employing a double tapered fiber configuration. It is known that as the fiber diameter decreases the light escapes from the fiber core and propagates in its cladding. The cladding coated with or made from thin fluorescent film guides the light due to total internal reflection at the cladding-air interface. At the second taper, where the fiber diameter increases, the light containing also the fluorescence signal re-couples back into the fiber core. Magnitude of the fluorescence signal extracted form the sensitive film by evanescent modes is proportional to the distance of light propagation through the polymer film. In the case of a double taper configuration the distance can be made as large as ten centimeters providing very high sensitivity. Removing of the fiber cladding and depositing the sensitive fluorescent material directly onto the fiber core allows further increasing the system sensitivity.

In additional preferred embodiment the sensitive fluorescent material is directly incorporated into the fiber-optic system playing a role of an optical waveguide. In that case the system sensitivity is enhanced by forcing the light to propagate through the sensitive material.

Thus, this method provides an efficient fluorescence-based means for highly sensitive and selective detection of the molecules of basic gases. As a general technology, the proposed method can find many useful applications for detecting different basic gases, including dimethyl methylphosphonate (DMMP) and such important chemical warfare agents as Sarin and Soman, as required for environmental monitoring, chemical industry and medicine.

Having described preferred embodiments of the invention with reference to the accompanying drawings, it is to be understood that the invention is not limited to the precise embodiments, and that various changes and modifications may be effected therein by skilled in the art without departing from the scope or spirit of the invention as defined in the appended claims.

What is claimed is:

1. A method of detecting molecules of basic gases employing at least one fluorescent sensitive material, comprising the steps of:
excitation of said at least one fluorescent sensitive material with at least one light source generating at the wavelengths required for efficient stimulating the fluorescence of said sensitive material,
collecting the fluorescent light signal from said fluorescent sensitive material with a light collecting system
detecting and processing the fluorescent light signal from said sensitive material at the wavelength maximizing the said fluorescent signal,
wherein said at least one fluorescent sensitive material comprises a hydrogen bond acidic polymer matrix and at least one solvatochromic dye with hydrogen bond basic groups isolated in said polymer matrix and said polymer matrix and said dye are chosen such to maximize the influence of the molecules of the basic gases under detection on said fluorescence light signal from said sensitive material.

2. A method of detecting molecules of basic gases of claim 1, wherein said dye and said polymer matrix are chosen such that the hydrogen-bond interaction between them results in depression of the fluorescence yield of said sensitive material prior its interaction with the molecules of basic gases and the interaction between the molecule of basic gases and said acidic polymer matrix breaks the hydrogen bond of said dye with said polymer matrix “releasing” said dye and returning it back into the state with a low solute-solvent interaction resulting in strong enhancement of the dye florescence quantum yield and shift of the dye fluorescent spectrum in the direction of shorter wavelengths.

3. A method of detecting molecules of basic gases of claim 1, wherein said step of detecting and processing the fluorescent light signal from said sensitive material includes detection and processing of both the said spectral shift of dye fluorescence and absorption spectrum maximizing the sensor sensitivity and selectivity to molecules of basic gases under detection.

4. A method of detecting molecules of basic gases of claim 1, wherein said sensitive fluorescent material is a film of Nile Red (NR) dye having functional basic group and isolated in BSP3 polymer matrix that is a strong hydrogen bond acidic polymer synthesized for sorption of basic gases, such as dimethyl methylphosphonate (DMMP), Sarin, Soman and other chemical warfare agents having basic properties.

5. A method of detecting molecules of basic gases of claim 4, wherein said wavelengths required for efficient stimulating the fluorescence of said sensitive material and said wavelength maximizing the said fluorescent signal are shifted to the shorter wavelengths with respect to the position of maximum of UV-Vis and fluorescence bands before DMMP exposure, such that NR interaction with DMMP leads to the blue shift of these spectra providing efficient amplification of the response signal.

6. A method of detecting molecules of basic gases of claim 1, wherein said at least one fluorescent sensitive material is incorporated into a waveguiding system delivering light from said at least one light source and effectively collecting the signal light from said fluorescent sensitive material.

7. A method of detecting molecules of basic gases of claim 6, wherein at least one section of said waveguiding system is made from said fluorescent sensitive material operating as a wavegude at wavelengths of said excitation light and said fluorescent signal light.

8. A method of detecting molecules of basic gases of claim 6, wherein said waveguiding system incorporates at least one optical fiber having at least one sharp U-bend and said fluorescent sensitive material having refractive index not less than the refractive index of the medium in which molecules of the basis gas under detection are present is deposited on outer surface of the fiber in the region of its said U-bend.

9. A method of detecting molecules of basic gases of claim 8, wherein cladding of said fiber in said outer region of U-bend is removed before depositing said fluorescent sensitive material improving excitation of the fluorescent sensitive materials and collecting the fluorescent signal light.

10. A method of detecting molecules of basic gases of claim 6, wherein said waveguiding system incorporates at least one optical fiber having at least one section with double tapered geometry characterized by decreasing and then increasing fiber diameter, said fluorescent sensitive material having refractive index not less than the refractive index of the medium in which molecules of the basis gas under detection are present is deposited on the surface of said fiber in its said double tapered region.

11. A method of detecting molecules of basic gases of claim 10, wherein cladding of said fiber in said double tapered region is removed before depositing said fluorescent sensitive material improving excitation of the fluorescent sensitive materials and collecting the fluorescent signal light.
